(19) United States
(12) Patent Application Publication
(10) Pub. No.: US 2024/0122671 A1
(43) Pub. Date: Apr. 18, 2024

(54) Computerized Systems for Arthroscopic Applications Using Real-Time Blood-Flow Detection

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian William QUIST, Salem, NH (US); Nathan Anil NETRAVALI, Littleton, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/277,972

(22) PCT Filed: Apr. 27, 2022
(86) PCT No.: PCT/US2022/026526

§ 371 Date: Aug. 18, 2023

Related U.S. Application Data
(60) Provisional application No. 63/180,734, filed on Apr. 28, 2021.

(51) Int. Cl. A61B 90/00; A61B 5/00; A61B 5/026; G16H 20/40; G16H 30/40

(52) U.S. Cl. CPC A61B 90/37; A61B 5/0022; A61B 5/0261; G16H 20/40; G16H 30/40

(57) ABSTRACT

Disclosed are systems and methods for a computerized framework that provides novel mechanisms for arthroscopic applications using real-time blood flow information. The disclosed framework operates by determining a real-time (or near real-time or substantially simultaneous) visualization of blood vessels or perfusion within anatomical structures during intraoperative procedures, and leveraging this determined information for the performance of an arthroscopic procedure. The disclosed framework can enable an arthroscopic camera to see blood flow and perfusion in tissues in real-time, which allows for differentiation of various parts of the anatomy that may otherwise be undetectable.

### CROSS-REFERENCE TO RELATED APPLICATIONS

This application claims the benefit of priority under 35 U.S.C. § 119(e) from U.S. Provisional Patent Application No. 63/180,734, filed Apr. 28, 2021, which is incorporated in its entirety herein by reference.

### TECHNICAL FIELD

The present disclosure generally relates to preoperative and intraoperative surgical data collection, analysis and processing, and more particularly, to real-time blood-flow detection and processing respective to vasculature information of a patient for conducting a surgical procedure.

### BACKGROUND

Navigational surgery systems typically include tracking devices and/or anatomy tracking via medical imaging. These systems are directed to identifying where a tool boundary or surface boundary of a particular anatomical structure is in three-dimensional (3D) space. Use of this type of information can be viewed as the core to the current set of robotic-enabled and/or navigation procedures, where precise bone removal and/or modification is performed with the aid of a computer assisted surgical system.

### SUMMARY

Tracking approaches for performing surgical procedures, therefore, are generally understood as an essential component of existing navigational surgery systems. However, conventional approaches typically fall short of providing the accurate and efficient processing required for many of today's medical procedures. For example, one of the challenges associated with arthroscopic procedures is that, based on the existing technology currently implemented by medical professionals, many of the tissues, especially the cartilage and bone, can appear uniform with no distinguishing features. This is one of the sub-optimal drawbacks from existing systems that can lead to prolonged surgical procedures, repeat procedures, and in some cases, inaccurate performance of a surgery which can have negative impacts on the patient, the medical staff and/or entity hosting the procedure (e.g., office, hospital, and the like).

For example, preoperative imaging, such as magnetic resonance imaging (MRI) or computed tomography (CT), can be used to capture anatomy, including bone, cartilage, ligaments, and other structures. These systems, however, are limited to the anatomical structures of a patient, and as provided above, can be limited in their applicability and overall effectiveness for a surgical procedure.

In another example, optical tracking systems (OTS) are the most commonly used tracking systems in commercial robotic platforms today. However, OTS require that a line-of-sight be maintained between the tracking device and the instrument to be tracked, which is not always possible in the operating theater, room or suite (OR) and potentially precludes tracking of surgical instruments inside the body. Indeed, such computer-aided surgery (CAS) systems not only require line-of-sight, but are also only accurate within a defined volume (respective to the camera position). As one of skill in the art would readily understand, this can be difficult to maintain throughout a surgical procedure, especially during manipulation of the patient's bony anatomy. OTS trackers also typically require additional skin incisions to be rigidly attached to bone.

In yet another example, electromagnetic (EM) navigation systems provide another commonly used tracking methodology, and it does not require the same line-of-sight or additional skin incisions as OTS. However, EM systems also suffer from a number of disadvantages. Similar to line-of-sight tracking, it can be difficult to maintain an optimal clinical workflow while also satisfying the requirements of the EM system. The EM system only provides accurate measurements within a defined volume, which is respective to a position of a field generator. Further, metal that may be commonly used during orthopedic and sports medicine procedures in the EM field, can generate interference and degrade the accuracy of the measurement.

Alternative approaches can involve mechanical tracking, which can be used via a mechanical arm or other similar structure being physically attached to the bone. These typically passive devices can determine motion by tracking each of the moving parts or joints between the end effector and the base of the device. However, much like the above example, they are deficient in accurately and effectively providing a least invasive surgical environment, as an appendage needs to be attached to a patient's anatomy, which can lead to additional complications not typically seen during conventional surgery.

The disclosed systems and methods provide a computerized framework that addresses current shortcomings in the existing technologies, inter alia, by providing novel mechanisms for arthroscopic applications using real-time blood flow information. According to some embodiments, the disclosed framework operates by determining a real-time (or near real-time or substantially simultaneous) visualization of blood vessels or perfusion within anatomical structures during intraoperative procedures, and leveraging this determined information for the performance of an arthroscopic procedure. As evident from the discussion herein, the disclosed framework can enable an arthroscopic camera to see blood flow and perfusion in tissues in real-time without requiring any dyes, which allows for differentiation of various parts of the anatomy that may i) otherwise appear similar via existing technologies and ii) may otherwise be undetectable via existing technologies.

It should be noted that while the discussion herein will focus on arthroscopic procedures (e.g., preparation and/or planning for, performance of, and evaluation afterwards, for example), it should not be construed as limiting, as any other type of known or to be known surgical and/or medical procedure that can benefit from the real-time determination of blood vessel visualizations and/or perfusion within anatomical structure can be subject to the disclosed systems and methods without departing from the scope of the instant disclosure.

According to some embodiments, the disclosed framework can operate by providing capabilities for visualization of a location of vasculature within anatomical structures, such that vascular structure can be leveraged to enable and/or guide surgical actions and/or decision making during the surgical procedures.

In some embodiments, the resulting blood vessels and/or blood flow information can be processed by a computing device monitoring the resulting image signal to provide an on-screen overlay with data/visualizations/information to support decision making by the surgeon intraoperative procedures.

In some embodiments, the vasculature identified can be used as a spatial reference for placement of virtual and/or digital location (or navigation) markers. For example, the anatomy of a patient can be assessed (e.g., analyzed) based on the blood flow information, whereby a virtual location marker(s) can be placed accordingly. In some embodiments, the assessment and/or placement of the marker(s) can be performed automatically by the disclosed framework, and in some embodiments, a surgeon can provide oversight and/or input that can trigger the marker's placement.

According to some embodiments, a virtual location marker may serve as a known point of return for later in the intraoperative procedure, or the virtual location marker may tag a location of action within the procedure, such as, for example, the location of an aperture drilled into the bone.

According to some embodiments, as an arthroscope is moved relative the anatomy during a surgical procedure, the disclosed framework can track the location of the virtual location marker(s) based on the vasculature shown by the blood flow (whether or not the vasculature is displayed on the display device). This enables the seamless point of point of reference analysis to be performed by the framework, thereby providing efficient and accurate maneuvering during the procedure.

In some embodiments, a patient's vasculature can be determined preoperatively using imaging with contrast (such as, computed tomography angiography (CTA) or magnetic resonance angiography (MRA), for example). In some embodiment, such computerized imagery can enable the creation of a preoperative model of the blood vessels and their positions and orientations relative to the anatomy. By using technologies that allows one to visualize the blood vessels and/or perfusion in real time intraoperatively, a “map” of the blood vessels can generated and used to register a preoperatively generated model of live images of the blood flow within the tissue.

According to some embodiments, a method is disclosed for determined visualization of a location of vasculature within anatomical structures, such that vascular structure can be leveraged to enable and/or guide surgical actions and/or decision making during the surgical procedures.

In accordance with one or more embodiments, the present disclosure provides a non-transitory computer-readable storage medium for carrying out the above mentioned technical steps. The non-transitory computer-readable storage medium has tangibly stored thereon, or tangibly encoded thereon, computer readable instructions that when executed by a device, cause at least one processor to perform a method for determined visualization of a location of vasculature within anatomical structures, such that vascular structure can be leveraged to enable and/or guide surgical actions and/or decision making during the surgical procedures.

In accordance with one or more embodiments, a system is provided that comprises one or more computing devices and/or apparatus configured to provide functionality in accordance with such embodiments. In accordance with one or more embodiments, functionality is embodied in steps of a method performed by at least one computing device and/or apparatus. In accordance with one or more embodiments, program code (or program logic) executed by a processor(s) of a computing device to implement functionality in accordance with one or more such embodiments is embodied in, by and/or on a non-transitory computer-readable medium.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary data flow according to some embodiments of the present disclosure;

FIG. 4 illustrates an exemplary data flow according to some embodiments of the present disclosure; and

FIG. 5 is a block diagram illustrating a computing device showing an example of a device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

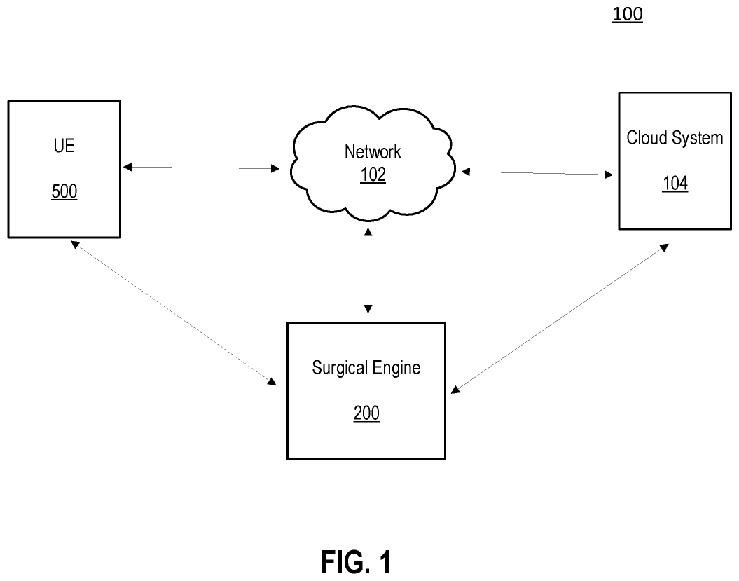

With reference to FIG. 1, system (or framework) 100 is depicted which includes UE 500 (e.g., a client device), network 102, cloud system 104 and surgical engine 200. UE 500 can be any type of device, such as, but not limited to, a mobile phone, tablet, laptop, personal computer, sensor, Internet of Things (IoT) device, autonomous machine, and any other device equipped with a cellular or wireless or wired transceiver.

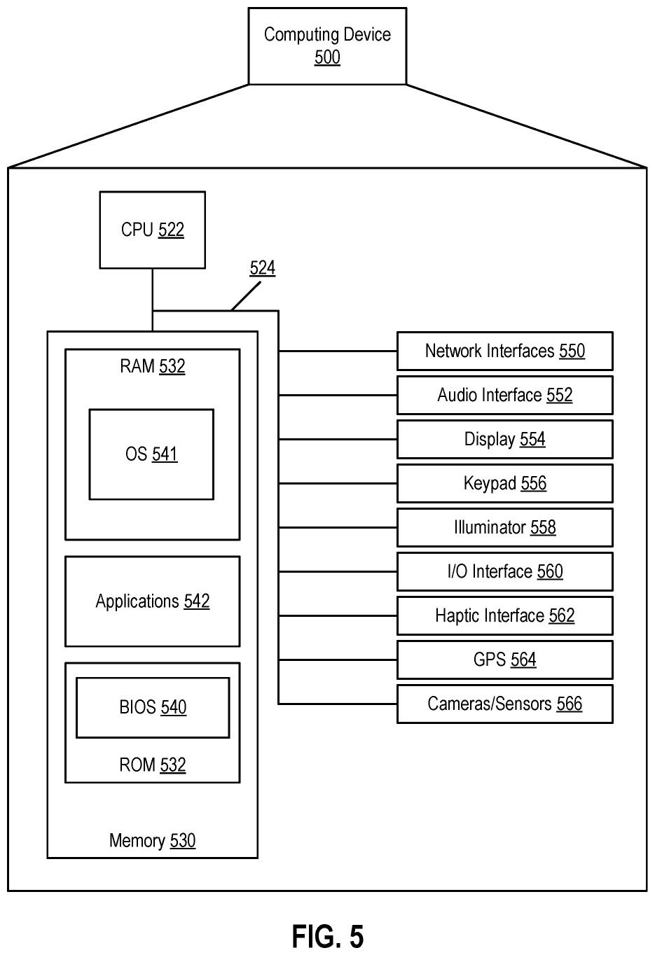

In some embodiments, as discussed above, UE 500 can also be a medical device, or another device that is communicatively coupled to a medical device that enables reception of readings from sensors of the medical device. For example, in some embodiments, UE 500 can be a neuromodulation device. In another example, in some embodiments, UE 500 can be a user's smartphone (or office/hospital equipment, for example) that is connected via WiFi, Bluetooth Low Energy (BLE) or NFC, for example, to a peripheral neuromodulation device. Thus, in some embodiments, UE 500 can be configured to receive data from sensors associated with a medical device, as discussed in more detail below. Further discussion of UE 500 is provided below at least in reference to FIG. 5.

Network 102 can be any type of network, such as, but not limited to, a wireless network, cellular network, the Internet, and the like (as discussed above). As discussed herein, network 102 can facilitate connectivity of the components of system 100, as illustrated in FIG. 1.

Cloud system 104 can be any type of cloud operating platform and/or network based system upon which applications, operations, and/or other forms of network resources can be located. For example, system 104 can correspond to a service provider, network provider and/or medical provider from where services and/or applications can be accessed, sourced or executed from. In some embodiments, cloud system 104 can include a server(s) and/or a database of information which is accessible over network 102. In some embodiments, a database (not shown) of system 104 can store a dataset of data and metadata associated with local and/or network information related to a user(s) of UE 500, patients and the UE 500, and the services and applications provided by cloud system 104 and/or surgical engine 200.

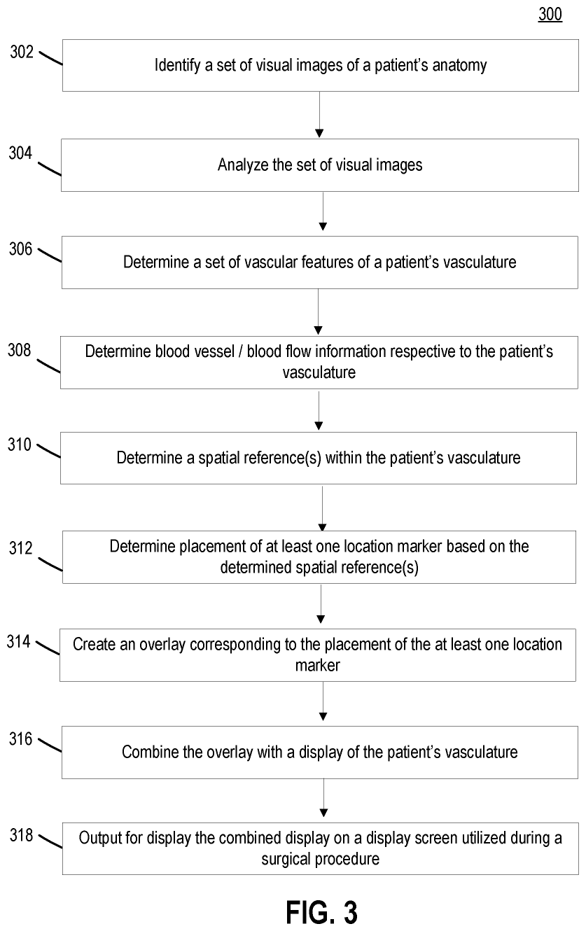

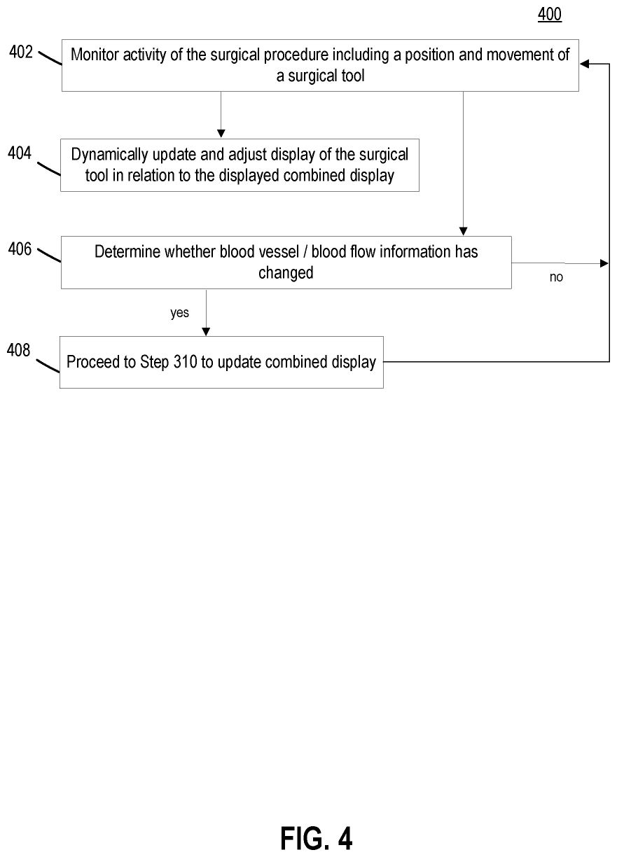

Surgical engine 200, as discussed below in more detail, includes components for determining a visualization of blood vessels or perfusion within anatomical structures during intraoperative procedures, and leveraging this determined information for the performance of an arthroscopic procedure. Moreover, engine 200 provides capabilities and functionality for real-time identification and/or determinations of a patient's vasculature information as a spatial reference for placement of virtual and/or digital location markers that stay in place as an arthroscope is moved relative the anatomy. Embodiments of how this is performed via engine 200, among others, are discussed in more detail below in relation to FIGS. 3-4.

According to some embodiments, surgical engine 200 can be a special purpose machine or processor and could be hosted by a device on network 102, within cloud system 104 and/or on UE 500. In some embodiments, engine 200 can be hosted by a peripheral device connected to UE 500 (e.g., a medical device, as discussed above).

According to some embodiments, surgical engine 200 can function as an application provided by cloud system 104. In some embodiments, engine 200 can function as an application installed on UE 500. In some embodiments, such application can be a web-based application accessed by UE 500 over network 102 from cloud system 104 (e.g., as indicated by the connection between network 102 and engine 200, and/or the dashed line between UE 500 and engine 200 in FIG. 1). In some embodiments, engine 200 can be configured and/or installed as an augmenting script, program or application (e.g., a plug-in or extension) to another application or program provided by cloud system 104 and/or executing on UE 500.

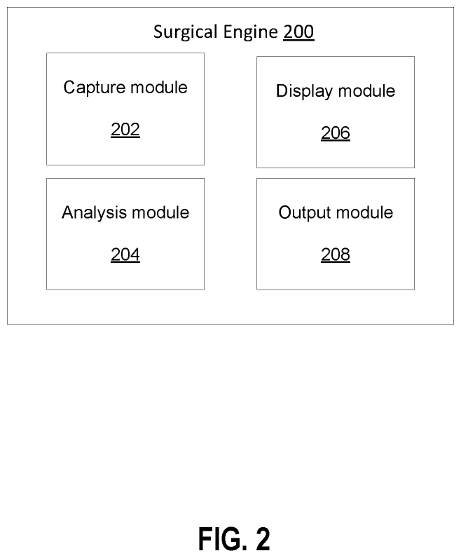

As illustrated in FIG. 2, according to some embodiments, surgical engine 200 includes capture module 202, analysis module 204, display module 206, output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 3, depicted is Process 300 which details non-limiting example embodiments of the disclosed framework's computerized operations for arthroscopic applications using real-time blood flow information. As discussed herein in relation to FIG. 3, the disclosed framework operates by determining a real-time (or near real-time or substantially simultaneous) visualization of blood vessels or perfusion within anatomical structures during intraoperative procedures, and leveraging this determined information for the performance of an arthroscopic procedure. According to some embodiments, as discussed below in relation to FIG. 4, the framework can enable an arthroscopic camera to see blood flow and perfusion in tissues in real-time, which allows for differentiation of various parts of the anatomy that may otherwise be undetectable.

According to some embodiments, Step 302 of Process 300 can be performed by capture module 202 of surgical engine 200; Steps 304-310 can be performed by analysis module 204; Steps 312-316 can be performed by display module 206; and Step 318 can be performed by output module 208.

Process 300 begins with Step 302 where engine 200 identifies a set of visual images of a patient's anatomy. In some embodiments, Step 302 can involve engine 200 triggering the capture of the visual images (e.g., engine 200 causes a medical device or UE 500 to capture the images); and, in some embodiments, Step 302 can involve engine 200 receiving captured image from a peripheral medical device, as discussed above.

According to some embodiments, the set of visual images can correspond to a video or image frames of a video that are captured by a medical device. In some embodiments, Step 302's image capture can be a live-stream or live-capture of a digital representation of the internal anatomy of a patient, in that they are currently being evaluated pre-operation (pre-op), during surgery and/or post-operation (post-op). That is, in some embodiments, engine 200 can execute the program logic associated with Step 302 so as to continually capture video image (e.g., image frames of a video) of the patient during the entirety of the processing of Process 300's steps (and in some embodiments, the processing of Process 400's steps (e.g., the monitoring of Step 402), as discussed below).

In Step 304, engine 200 analyzes the images identified in Step 302. According to some embodiments, the computational analysis performed in Step 302 can involve engine 200 executing any type of known or to be known machine learning (ML) or artificial intelligence (AI) computational analysis algorithm, technology, mechanism or classifier, such as, but not limited to, neural networks (e.g., artificial neural network analysis (ANN), convolutional neural network (CNN) analysis, and the like), computer vision, cluster analysis, data mining, Bayesian network analysis, Hidden Markov models, logical model and/or tree analysis, and the like.

In Step 306, based on the computational analysis performed by engine 200 in Step 304, engine 200 can parse the identified set of images, and determine, derive, extract or otherwise identify a set of vascular features of a patient's vasculature. According to some embodiments, Step 306 can involve the determination of a network of blood vessels connecting the heart with other organs and tissues of the body. In some embodiments, engine 200 can identify the vasculature for an entire body or a portion of the body (e.g., the portion that corresponds to the surgical site). In some embodiments, the vascular features can correspond to information related to, but not limited to, types of arteries, arterioles, venules, veins and capillaries; structure, materials and/or consistency of particular tissue(s); anatomical structure of parts of the body; and the like, or some combination thereof.

According to some embodiments, Step 306 can involve engine 200 creating or generating a user interface (UI) based on the determined set of vascular features. In some embodiments, each vascular feature can be displayable within the UI as an information object (IO). In some embodiments, vascular UI can include information related to, but not limited to, blood vessel/flow information, vascular features, and the like, or some combinations thereof. Thus, in some embodiments, identified set of vascular features themselves, as IOs within a displayed UI, can serve as trackable digital markers, as discussed herein and in more detail below.

In Step 308, engine 200 can determine blood vessel and/or blood flow (or blood-flow, used interchangeably) information respective to the patient's vasculature. According to some embodiments, engine 200 can perform this determination based on the computational analysis performed in Step 304 (e.g., in a similar manner as discussed above in relation to at least Step 306). In some embodiments, Step 304 can be re-executed by engine 200 in order to specifically determine the blood vessel/flow information, as discussed herein.

According to some embodiments, the determined blood vessel/blood flow information can include information related to, but not limited to, blood flow volume and velocity of particular arteries, arterioles, venules, veins, capillaries and tissues; oxygen delivery levels; nutrient delivery levels; and the like, or some combination thereof.

In Step 310, a spatial reference(s) within the patient's vasculature is determined. In some embodiments, the spatial reference is based on the determined vascular features from Step 306 and the blood vessel/blood flow information from Step 308. According to some embodiments, the information determined from Steps 306 and 308 can be input into a ML/AI algorithm (as discussed above in relation to Step 304), as a result, spatial reference information can be determined.

According to some embodiments, spatial reference information can correspond to, but is not limited to, specific parts of the patient's body related to a surgical procedure, specific parts of the patient's body to avoid (e.g., a part of tissue that may cause unnecessary blood loss should it be cut, for example), regions of the body, and the like, or some combination thereof. In some embodiments, the spatial reference can be configured as a 3D or 2D modelling, or an n-dimensional feature vector that outlines attributes of the patient's body with particular features and information corresponding to nodes on the vector.

According to some embodiments, spatial references can be subject to a threshold distance so that the area in and/or around the reference area indicates an area within the patient's vasculature that is to be focused on and/or avoided, as mentioned above.

For example, according to some embodiments, engine 200 can leverage the determined knowledge of the patient's vascular structure (from Step 306) and the knowledge of how the patient's blood flows via vessels in and around such structure (from Step 308) to identify spatial reference points (or spatial reference information) that can be utilized to aid in the planning and execution of implant placement, as discussed below. For example, certain implants may benefit from proximity to vasculature, especially those made of a bioabsorbable material. By placing them proximal to or through vasculature, they may be more readily replaced by native tissue. However, other implants may benefit from being placed away from vasculature as they may impede the tissue's natural ability to function and heal or cause unnecessary blood loss. Identification of such spatial reference locations within the patient can improve how the surgeon performs the operation in that the surgeon is afforded the knowledge of which vascular structure to avoid and/or which can be interacted with.

In Step 312, engine 200 determines a placement of at least one location marker based on the determined spatial reference(s). In some embodiments, the number of unique markers can directly correspond to a number of unique spatial references. In some embodiments, a spatial reference can have associated therewith a plurality of markers so as to delineate the proximity to focus on and/or avoid within the vascular structure of the patient. According to some embodiments, the location markers, as discussed below, can serve as a target for an item (e.g., a location for drilling a tunnel or placing an implant, for example), and/or can be used to track a tool's position relative to the anatomy.

According to some embodiments, the location markers can be configured as digital tags or items and/or virtual tags or items. In some embodiments, the tags or items can be configured as displayable IOs on a display screen or UI, as discussed below. In some embodiments, the IOs can be displayable as part of an augmented reality (AR) or virtual reality (VR) display.

In some embodiments, each digital/virtual marker can, but is not limited to, be uniquely identified (e.g., have a specific identifier (ID)), be subject to a privacy enhancing technology (PET) or security enhancing technology (SET), indicate values, shapes, sizes and/or patterns related to a vascular feature and/or blood flow quality/characteristic, and the like, or some combination thereof. As mentioned above, the digital markers can correspond to the set of vascular features.

In Step 314, engine 200 can create an overlay display UI. The created overlay UI can include information related to the IOs for each determined location marker. According to some embodiments, the UI can include, but is not limited to, information related to each determined location marker, spatial reference information, blood vessel/flow information, vascular features, and the like, or some combinations thereof.

In Step 316, engine 200 combines the overlay display UI (from Step 314) with the vascular UI (from Step 306). In some embodiments, Step 316 can involve the creation of another UI (e.g., a new UI that includes the information from the overlay display UI and the vascular UI). In some embodiments, Step 316's UI can involve engine 200 modifying the vascular UI to include and display characteristics/attributes of the overlay display UI (and vice versa).

Thus, according to some embodiments, Step 316 involves engine 200 generating a combined UI that displays the at least one location marker in accordance with the vascular features of the patient.

In some embodiments, the combined UI generated in Step 316 can be any type of displayable object(s) or item(s), including, but not limited to, an UI, VR display, AR display, and the like. For example, a 3D anatomical model can be generated (e.g., via engine 200 executing statistical shape modeling or atlas based modeling or other similar mechanisms based on the vascular features and the blood vessel/blood flow information).

Some embodiments may exist, however, where given the high-resolution and interactive nature of the combined UI display (e.g., a VR display of the patient's anatomy and blood information), location markers may not be needed as the pulse rate, blood flow and location of such tissue can be readily displayed and indicated, thereby negating a need for such location markers.

In Step 318, engine 200 outputs the combined UI for display on a display screen that is or can be used during a surgical procedure. This enables a medical professional (e.g., surgeon) to capture and track their movements during a procedure to avoid particular elements of a patient, while being able to more accurately perform the procedure. Embodiments of how this display can be utilized are discussed in more detail below in relation to FIG. 4 and Process 400.

According to some embodiments, each of the information determined, created, and the output from the analysis of Process 300 can be stored in an electronic medical record (EMR) for the patient. In some embodiments, this information can be fed to the ML/AI algorithms discussed above for further training and refinement of their accuracy and efficiency in identifying and determined specifically required surgical information discussed above. Similarly, the information monitored, analyzed, determined and output respective to the steps of Process 400 discussed below can be stored in the EMR and fed to the ML/AI algorithms.

Turning to FIG. 4, Process 400 details the implementation of the combined display from Process 300, as discussed above. According to some embodiments, Process 400 provides non-limiting example embodiments of engine 200's operation and implementation during a surgical procedure that can enable advanced and improved surgical accuracy and effectiveness. That is, by turning on the visualization of the blood flow/vessels, the video/images can be captured and displayed in real-time. In some embodiments, by placing a marker within the tissue with a known visual pattern on it or via tracking using any type of known or to be known tracking technology, the positions and orientations of the vascular structure can then be determined and registered to the marker. Then, as discussed below, as the marker changes position and orientation with respect to the camera, the patient's tissue can be tracked throughout the procedure.

According to some embodiments, engine 200, a surgeon (or other medical professional involved in the procedure), and/or any other type of application or device that controls image capture capabilities being used for a procedure, can trigger a live update of blood flow. In some embodiments, based on a placement of a location marker was used, a camera can then be moved but the on-screen navigation may remain fixed to the anatomy of the patient. In some embodiments, the live blood flow could be used to generate a structural representation used to re-register the guidance. In this way, the guidance would continually be ‘pulsed’ to provide updates to the surgeon on both guidance and navigation overlay relative to the patient's anatomy.

According to some embodiments, Steps 402 and 406-408 can be performed by analysis module 204 of surgical engine 200; and Step 404 can be performed by display module 206.

Process 400 begins with Step 402 where engine 200 monitors activity (or status) of the surgical procedure including a position and movement of a surgical tool. The monitoring can be performed during a visualization depicted via the combined UI display (from Step 318, supra). In some embodiments, Step 402 can further involve engine 200 executing any type of known or to be known noise reduction algorithm to reduce noise and enhance the vascular features depicted in the combined UI display.

Process 400 proceeds to Step 404 where, as the surgeon manipulates the surgical tools and/or based on camera movements, as discussed above, engine 200 can determine to dynamically update and adjust the display of the combined display. In some embodiments, engine 200 can detect position changes of the camera respective to the fixed position of the location marker, and adjust the display accordingly. An example of this is the pulsed guidance discussed above, whereby perspectives of the patient's anatomy are adjusted based on a camera's position and perspective relative to the location marker depicted in the visualized combined UI display.

Process 400 can also proceed from Step 402 to Step 406 where engine 200 determines whether blood vessel/blood flow information has changed during the monitoring of the surgical procedure. This determination can enable an accurate, up-to-date (e.g., real-time) indication of which tissue to avoid and/or target, as discussed above.

In some embodiments, when engine 200 determines that the blood vessel/blood flow information has not changed (e.g., at least a threshold amount or has stayed within a threshold range of an initial/previous value/measure), Process 400 can proceed to from Step 406 back to Step 402 for continued monitoring of the procedure based on existing blood vessel/blood flow values. This, therefore, enables the procedure to proceed with existing combined UI information (or overlay UI information determined from Process 300, as discussed above).

In some embodiments, when engine 200 determines that the blood vessel/blood flow information has changed (e.g., at least a threshold amount or as changed values outside a threshold range from the initial/previous value/measure), Process 400 can proceed from Step 406 to Step 408. In Step 408, engine 200 executes Step 310 of Process 300 (and the subsequent steps of Step 310 within Process 300) so as to provide an updated combined UI (or at least an updated overlay UI). Upon the completion of Step 408, Process 400 then proceeds back to Step 402 where monitoring of the procedure is performed via the updated combined UI.

According to some embodiments, by way of additional information to characterize non-limiting implementations of the disclosed framework, engine 200 can be leveraged for procedures including, but not limited to, soft tissue repair, meniscal repair, hip labral repair, shoulder labral repair, microfracture, anchor placement, bone healing, and the like.

For example, meniscal tears that are fully in the avascular or “white zone” are typically not repaired due to the lack of blood supply and their unlikelihood of healing, whereas meniscal tears in the vascular or “red zone” likely have the ability to heal. There is a transitional zone referred to as the “red-white zone,” in which there may be a potential to heal but the vasculature is traditionally difficult to see intraoperatively. By implementing the disclosed framework's capabilities and functionality, visualization of the vasculature in real-time intraoperatively can be provided, which can enable a more informed decision with regards to repairing the meniscus.

In another non-limiting example, blood supply of the acetabular labrum as provided from the periacetabular periosteal vascular ring can be utilized for healing of the acetabular labrum after repair; and, the disclosed framework's capabilities can enable such blood supply leveraging.

In yet another non-limiting example, in a patient's shoulder, the areas between the anterior and superior labrum have a limited blood supply as compared to the inferior labrum. Further, the outside areas of the labrum have more of a blood supply than the central portion. Having intraoperative information of a patient's specific vasculature via the disclosed framework's implementation may better inform the surgeon around how to perform a labral repair.

Similarly, cartilage can have limited healing capabilities due to a limited vascular supply. Microfracture is a procedure in which a small sharp pick is used to create holes within the bone at the base of an articular cartilage defect to release blood such that a clot can form and fibrocartilage can fill in the defect. By visualizing the blood flow intraoperatively via implementation of the disclosed framework, the creation of holes can potentially be directed more effectively to release more blood with fewer holes.

With regard to an anchor placement procedure, when reattaching soft tissue to bone (e.g., labral repair of the hip or rotator cuff repair), it is important to ensure that the suture anchors are placed in good quality bone. Placing the anchors near vasculature via implementation of the disclosed framework can enhance the likelihood of these anchors remaining secure as the surrounding bone heals after the procedure.

And, with regard to bone healing procedures, such as, for example, a Latarjet procedure, where significant bone loss in the glenoid is replaced by the coracoid to try and restore a more native geometry and reduce the chance of dislocation, once the coracoid is removed, it may be beneficial to expose portions of the glenoid that have good vasculature such that the fusion between the coracoid and glenoid is enhanced. As evidenced from the discussion herein, implementation of the disclosed framework can effectuate such beneficial advantages for bone healing procedures.

Thus, according to some embodiments, the disclosed framework can be utilized to suggest areas for placement of implants or microfracture, and/or provide warnings to a surgeon that certain areas may not have enough blood supply for certain elements of the procedure that they are performing. Moreover, the disclosed technology can enhance surgical navigation and robotics procedures by providing additional information of the anatomy that may not be traditionally visible. This can be used for registration of anatomy to preoperative or intraoperatively generated anatomic models or even for tracking anatomy without the need for more traditional markers. This reduces the risk of damage or complications since no holes need to be placed in the anatomy and there is no risk of crushing any structures with a clamp. Further, the lack of needing to place any markers within the anatomy will save time compared to other methods that require this step. This additional information of the vascular can greatly speed up the registration and tracking process as compared to using any other type of conventional trackers.

FIG. 5 is a block diagram illustrating a computing device 500 (e.g., UE 500, as discussed above) showing an example of a client device or server device used in the various embodiments of the disclosure.

The computing device 500 may include more or fewer components than those shown in FIG. 5, depending on the deployment or usage of the device 500. For example, a server computing device, such as a rack-mounted server, may not include audio interfaces 552, displays 554, keypads 556, illuminators 558, haptic interfaces 562, GPS receivers 564, or cameras/sensors 566. Some devices may include additional components not shown, such as GPU devices, cryptographic co-processors, AI accelerators, or other peripheral devices.

As shown in FIG. 5, the device 500 includes a central processing unit (CPU) 522 in communication with a mass memory 530 via a bus 524. The computing device 500 also includes one or more network interfaces 550, an audio interface 552, a display 554, a keypad 556, an illuminator 558, an input/output interface 560, a haptic interface 562, an optional GPS receiver 564 (and/or an interchangeable or additional GNSS receiver) and a camera(s) or other optical, thermal, or electromagnetic sensors 566. Device 500 can include one camera/sensor 566 or a plurality of cameras/sensors 566. The positioning of the camera(s)/sensor(s) 566 on the device 500 can change per device 500 model, per device 500 capabilities, and the like, or some combination thereof.

In some embodiments, the CPU 522 may comprise a general-purpose CPU. The CPU 522 may comprise a single-core or multiple-core CPU. The CPU 522 may comprise a system-on-a-chip (SoC) or a similar embedded system. In some embodiments, a GPU may be used in place of, or in combination with, a CPU 522. Mass memory 530 may comprise a dynamic random-access memory (DRAM) device, a static random-access memory device (SRAM), or a Flash (e.g., NAND Flash) memory device. In some embodiments, mass memory 530 may comprise a combination of such memory types. In one embodiment, the bus 524 may comprise a Peripheral Component Interconnect Express (PCIe) bus. In some embodiments, the bus 524 may comprise multiple busses instead of a single bus.

Mass memory 530 illustrates another example of computer storage media for the storage of information such as computer-readable instructions, data structures, program modules, or other data. Mass memory 530 stores a basic input/output system (“BIOS”) 540 for controlling the low-level operation of the computing device 500. The mass memory also stores an operating system 541 for controlling the operation of the computing device 500.

Applications 542 may include computer-executable instructions which, when executed by the computing device 500, perform any of the methods (or portions of the methods) described previously in the description of the preceding Figures. In some embodiments, the software or programs implementing the method embodiments can be read from a hard disk drive (not illustrated) and temporarily stored in RAM 532 by CPU 522. CPU 522 may then read the software or data from RAM 532, process them, and store them to RAM 532 again.

The computing device 500 may optionally communicate with a base station (not shown) or directly with another computing device. Network interface 550 is sometimes known as a transceiver, transceiving device, or network interface card (NIC).

The audio interface 552 produces and receives audio signals such as the sound of a human voice. For example, the audio interface 552 may be coupled to a speaker and microphone (not shown) to enable telecommunication with others or generate an audio acknowledgment for some action. Display 554 may be a liquid crystal display (LCD), gas plasma, light-emitting diode (LED), or any other type of display used with a computing device. Display 554 may also include a touch-sensitive screen arranged to receive input from an object such as a stylus or a digit from a human hand.

Keypad 556 may comprise any input device arranged to receive input from a user. Illuminator 558 may provide a status indication or provide light.

The computing device 500 also comprises an input/output interface 560 for communicating with external devices, using communication technologies, such as USB, infrared, Bluetooth™, or the like. The haptic interface 562 provides tactile feedback to a user of the client device.

The optional GPS transceiver 564 can determine the physical coordinates of the computing device 500 on the surface of the Earth, which typically outputs a location as latitude and longitude values. GPS transceiver 564 can also employ other geo-positioning mechanisms, including, but not limited to, triangulation, assisted GPS (AGPS), E-OTD, CI, SAI, ETA, BSS, or the like, to further determine the physical location of the computing device 500 on the surface of the Earth. In one embodiment, however, the computing device 500 may communicate through other components, provide other information that may be employed to determine a physical location of the device, including, for example, a MAC address, IP address, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

For the purposes of this disclosure the term “user”, “data owner”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

1. A method comprising the steps of:
identifying, by a device, a set of visual images of a patient's anatomy;
analyzing, by the device, the set of visual images;
determining, by the device, based on the analysis of the set of visual images, a set of vascular features associated with a vasculature of the patient;
further determining, by the device, based on the analysis of the set of visual images, blood information respective to the vasculature, the blood information corresponding to a blood flow of the vasculature;
determining, by the device, a placement of a digital location marker respective to the patient's anatomy, the placement determination based on the set of vascular features and the blood information; and
generating, by the device, a user interface (UI) that based on the determined placement of the digital location marker, the set of vascular features and the blood information.

2. The method of claim 1, further comprising:
outputting for display on a display of a device within an operating theater (OR) the generated UI, the output display comprising a display of the digital location marker at a fixed position corresponding to the determined placement, and indications of the set of vascular features and the blood information.

3. The method of claim 2, further comprising:
monitoring activities of a surgical procedure via the output display of the generated UI;
detecting, based on the monitoring, position changes of a surgical tool respective to the digital location marker; and
adjusting a perspective of the output display of the UI while maintaining the fixed position of the digital location marker.

4. The method of claim 3, further comprising:
determining, based on the monitoring, whether the blood information for the patient has changed, wherein:
the device re-performs the determination of the blood information step to update the generated UI being displayed when the blood information is determined to have changed at least a threshold amount, and
the device continues the monitoring the activities of the surgical procedure according to the generated UI when the blood information is determined to not have changed at least the threshold amount.

5. The method of claim 1, further comprising:
determining a spatial reference within the patient's vasculature, the spatial reference corresponding to a particular tissue identifiable from the blood information, the spatial reference being in reference to vasculature of the patient,
wherein the determination of the placement of the digital location marker is further based on the determined spatial reference.

6. The method of claim 1, further comprising:
generating a vascular UI based on the set vascular features; and
generating an overlay UI based on the blood information,
wherein the generated UI is based on the vascular UI and the overlay UI.

7. The method of claim 1, wherein the set of vascular features correspond to information selected from a group consisting of: arteries, arterioles, venules, veins and capillaries; structure, materials and/or consistency of particular tissue; and anatomical structure of parts of the patient's body.

8. The method of claim 1, wherein the set of vascular features correspond to a specific portion of the patient for which a surgical procedure will be performed.

9. The method of claim 1, further comprising:
determining, by the device, a placement of a set of digital location markers based on the set of vascular features, wherein the UI is generated based on the set of digital location markers.

10. The method of claim 1, wherein the blood information comprises information selected from a group consisting of: blood flow volume and velocity of particular arteries, arterioles, venules, veins, capillaries and tissues; oxygen delivery levels; and nutrient delivery levels.

11. A non-transitory computer-readable storage medium tangibly encoded with computer-executable instructions, that when executed by a device, perform a method comprising steps of:
identifying, by the device, a set of visual images of a patient's anatomy;
analyzing, by the device, the set of visual images;
determining, by the device, based on the analysis of the set of visual images, a set of vascular features associated with a vasculature of the patient;
further determining, by the device, based on the analysis of the set of visual images, blood information respective to the vasculature, the blood information corresponding to a blood flow of the vasculature;
determining, by the device, a placement of a digital location marker respective to the patient's anatomy, the placement determination based on the set of vascular features and the blood information; and
generating, by the device, a user interface (UI) that based on the determined placement of the digital location marker, the set of vascular features and the blood information.

12. The non-transitory computer-readable storage medium of claim 11, further comprising:
outputting for display on a display of a device within an operating theater (OR) the generated UI, the output display comprising a display of the digital location marker at a fixed position corresponding to the determined placement, and indications of the set of vascular features and the blood information;
monitoring activities of a surgical procedure via the output display of the generated UI;
detecting, based on the monitoring, position changes of a surgical tool respective to the digital location marker; and
adjusting a perspective of the output display of the UI while maintaining the fixed position of the digital location marker.

13. The non-transitory computer-readable storage medium of claim 12, further comprising:
determining, based on the monitoring, whether the blood information for the patient has changed, wherein:
the device re-performs the determination of the blood information step to update the generated UI being displayed when the blood information is determined to have changed at least a threshold amount, and
the device continues the monitoring the activities of the surgical procedure according to the generated UI when the blood information is determined to not have changed at least the threshold amount.

14. The non-transitory computer-readable storage medium of claim 11, further comprising:
determining a spatial reference within the patient's vasculature, the spatial reference corresponding to a particular tissue identifiable from the blood information, the spatial reference being in reference to vasculature of the patient,
wherein the determination of the placement of the digital location marker is further based on the determined spatial reference.

15. The non-transitory computer-readable storage medium of claim 11, further comprising:
generating a vascular UI based on the set vascular features; and
generating an overlay UI based on the blood information,
wherein the generated UI is based on the vascular UI and the overlay UI.

16. The non-transitory computer-readable storage medium of claim 11, further comprising:
determining, by the device, a placement of a set of digital location markers based on the set of vascular features, wherein the UI is generated based on the set of digital location markers.

17. A device comprising:
a processor configured to:
identify a set of visual images of a patient's anatomy;
analyze the set of visual images;
determine, based on the analysis of the set of visual images, a set of vascular features associated with a vasculature of the patient;
further determine, based on the analysis of the set of visual images, blood information respective to the vasculature, the blood information corresponding to a blood flow of the vasculature;
determine a placement of a digital location marker respective to the patient's anatomy, the placement determination based on the set of vascular features and the blood information; and
generate a user interface (UI) that based on the determined placement of the digital location marker, the set of vascular features and the blood information.

18. The device of claim 17, wherein the processor is further configured to:
output for display on a display of a device within an operating theater (OR) the generated UI, the output display comprising a display of the digital location marker at a fixed position corresponding to the determined placement, and indications of the set of vascular features and the blood information;
monitor activities of a surgical procedure via the output display of the generated UI;
detect, based on the monitoring, position changes of a surgical tool respective to the digital location marker; and
adjust a perspective of the output display of the UI while maintaining the fixed position of the digital location marker.

19. The device of claim 18, wherein the processor is further configured to:
determine, based on the monitoring, whether the blood information for the patient has changed, wherein:
the device re-performs the determination of the blood information step to update the generated UI being displayed when the blood information is determined to have changed at least a threshold amount, and
the device continues the monitoring the activities of the surgical procedure according to the generated UI when the blood information is determined to not have changed at least the threshold amount.

20. The device of claim 17, further comprising:
determine a placement of a set of digital location markers based on the set of vascular features, wherein the UI is generated based on the set of digital location markers.
